(19) United States
(12) Patent Application Publication
(10) Pub. No.: US 2002/0099349 A1
(43) Pub. Date: Jul. 25, 2002

(54) Men's underwear napikn

(76) Inventor: Hsin Fa Lee, Kaohsiung (TW)

Correspondence Address: Keith Kline, PRO-TECHTOR INTERNATIONAL SERVICES, 20775 Norada Court, Saratoga, CA, 95070-3018, US

(21) Appl. No.: 09/836,121

(22) Filed: Apr. 16, 2001

(30) Foreign Application Priority Data
Jan. 19, 2001 (TW) 90201135

(51) Int. Cl. A61F 13/15; A61F 13/20

(52) U.S. Cl. 604/385.03; 604/387

(57) ABSTRACT

A napkin for man's briefs or shorts that has one adhesive side to be adhered to a crotch inside the briefs and the other side comprised of a soft and absorbent layer that directly contact the man's penis to absorb any residual urine remained in the external urethral orifice after urination or has its adhesive side adhered in the briefs at where facing the anus to maintain the briefs clean and comfortable for personal hygiene purpose.

### BACKGROUND OF THE INVENTION

(a) Field of the Invention

The present invention is to provide men's underwear napkin, and more particularly, to one that effectively prevents direct contamination of residual urine in briefs of men's underwear for personal hygiene and comfort.

(b) Description of the Prior Art

Whereas physical difference in reproduction organs exits between both genders, a female tends to wipe her external urethral orifice with a tissue after urination. However, a male do not develop such habit of using a tissue to clean his external urethral orifice due to less privacy or for cause whatsoever. Usually, a slight shaking of his penis will consider sufficient to get rid off residual urine. However, residual urine sustains in his briefs.

For a senile male who suffers polyurea and/or diabetes is likely to sustain residual urine after urination and is vulnerable to get his briefs sipped by the residual urine. He has least chance to wear clean and comfortable briefs. Besides, the stains caused by the residual on the briefs are very stubborn in laundry and a bacteria affection problem is threatening.

### SUMMARY OF THE INVENTION

The primary purpose of the present invention is to provide men's underwear napkin to effectively improved the problem of contaminated briefs and give the man to wear clean and comfort briefs.

### BRIEF DESCRIPTION OF THE DRAWINGS

FIG. 1 is a schematic view of a preferred embodiment of the present invention placed inside men's briefs.

FIG. 2 is a schematic view showing how to use a self-adhesive napkin of the preferred embodiment of the present invention.

FIG. 3 is a schematic view showing how to wear the preferred embodiment of the present invention.

FIG. 4 is a schematic view showing the preferred embodiment of the present invention applied in a pair of boxer shorts.

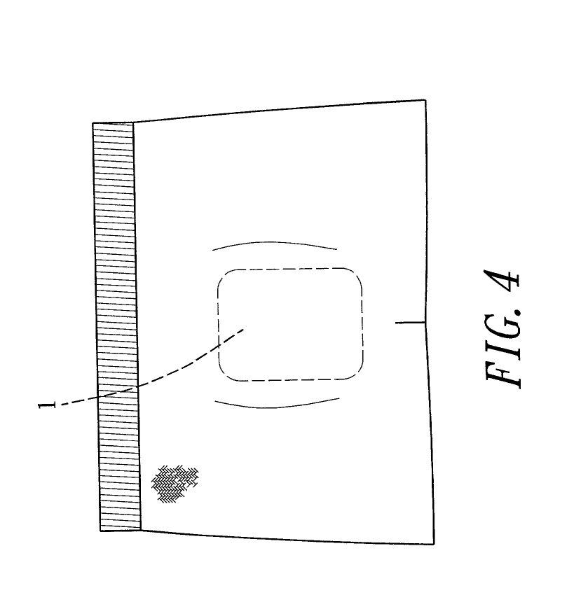

FIG. 5 is a schematic view showing a position of the preferred embodiment of the present invention in relation to anus.

### DETAILED DESCRIPTION OF THE PREFERRED EMBODIMENTS

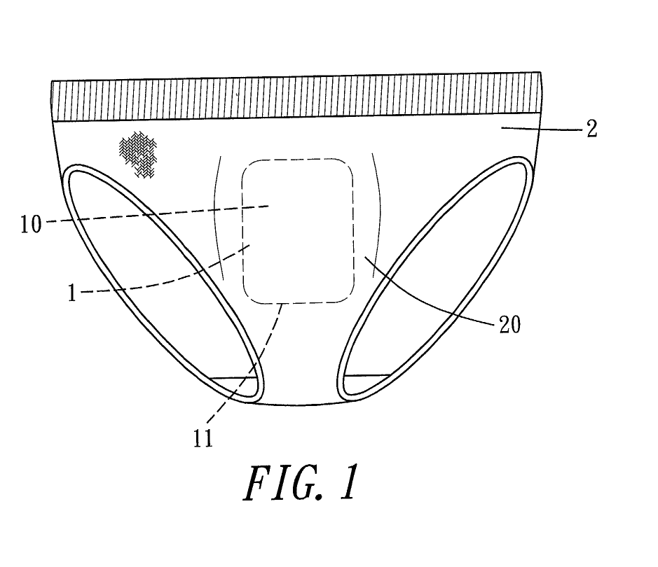

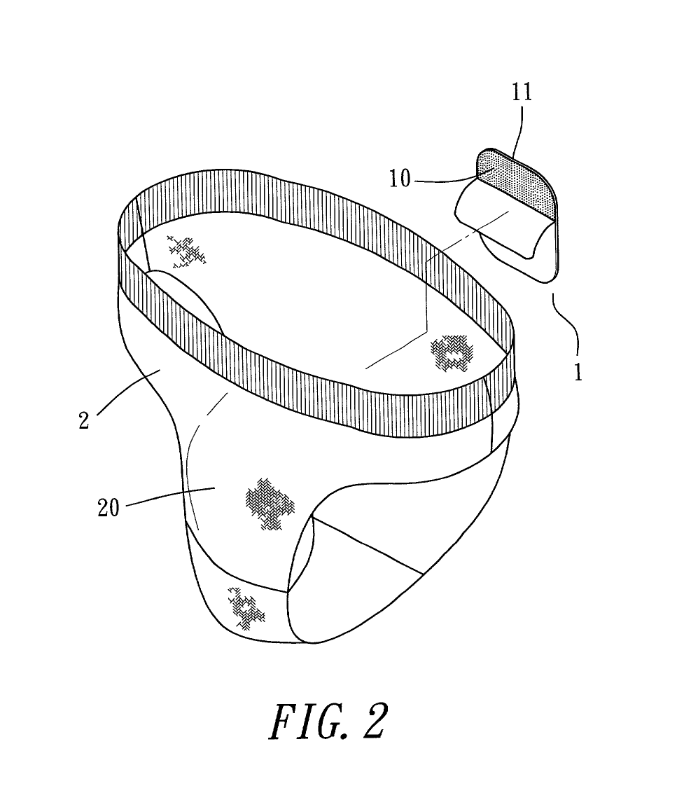

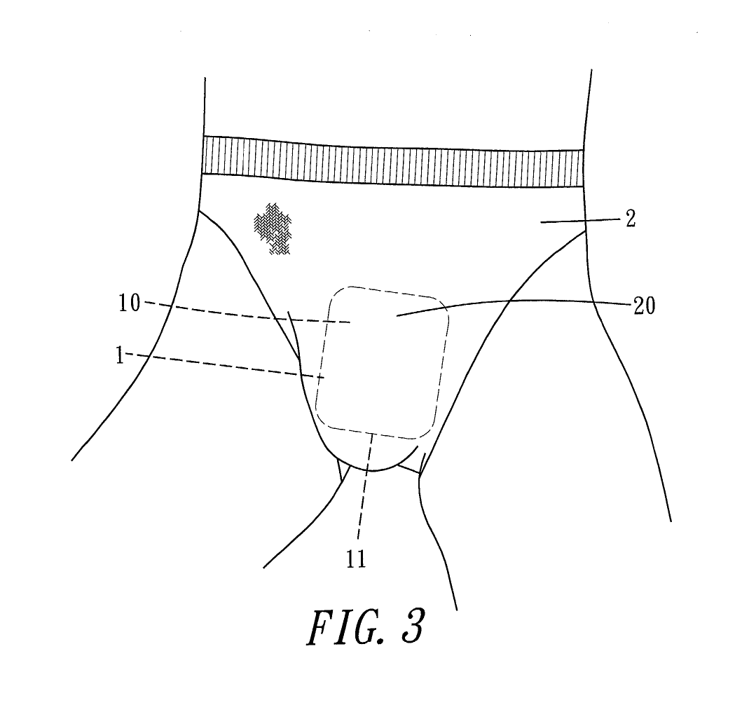

Referring to FIG. 1, it shows a schematic view of a napkin I exclusively for wearing inside men's briefs 2. Wherein, one side of said napkin 1 relates to a tear-and-use adhesive surface 10 while the other side to a soft, absorbent napkin layer 11. Said adhesive surface 10 is used to stick to where inside a crotch 20 of the briefs 2 as illustrated in FIG. 2 leaving the other side of napkin layer 11 directly contact a reproduction organ of a man. Upon wearing said briefs 2 attached with the napkin 1 as illustrated in FIG. 3, a mild and comfortable touch is delivered by the napkin layer 11 to the man's reproduction organ. In addition, even there is residual urine after urination, such residual urine can be absorbed by the napkin layer to maintain the briefs always in clean and comfortable conditions, thus to achieve the purpose of personal hygiene.

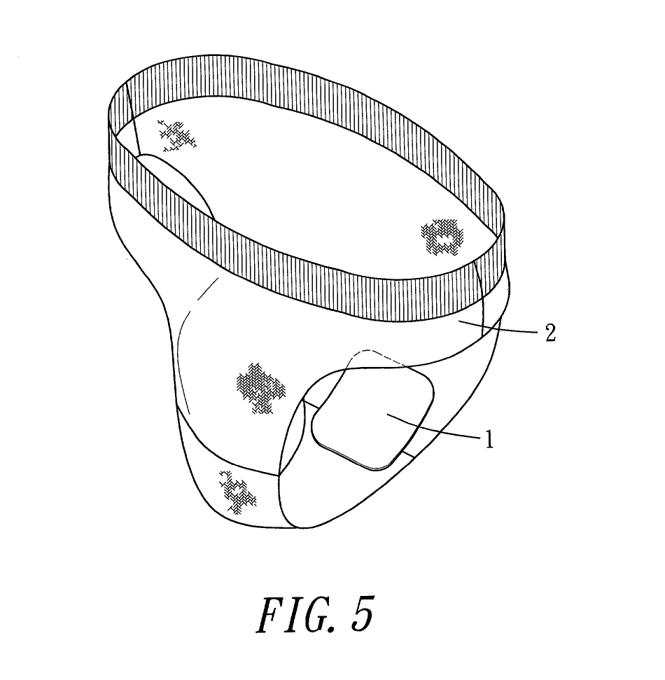

Furthermore, in terms of use pattern, said napkin 1 can be also attached inside the briefs 2 at where with its adhesive side facing where the briefs 2 contacts the anus as illustrated in FIG. 5. This is a position of application of the napkin particularly beneficial for the man either suffers hemorrhoids or who is very self-demanding in personal hygiene.

Consequently, the present invention provides the man many advantages including but not limited to the follows:

(1) To a man suffers polyurea and/or diabetes, the present invention effectively releases him from feeling uncomfortable and poor personal hygiene problems due to that his briefs has been contaminated by residual urine.

(2) To a teen-ager recovered from a wet dream will have to frequently change his briefs and do the laundry. With the present invention, he has only to replace the napkin after any occasional wet dream.

(3) The present invention meets hygiene requirements by preventing the briefs from being contaminated by any residual urine.

(4) More or less, dirt from residual urine sustains and is very difficult to clean in a laundry machine. Therefore, either using strong bleaching agent or simply buy a new pair of briefs. With the present invention, the napkin prevents any residual urine from contacting and thus contaminating the briefs. Therefore, the present invention helps service life of a pair of briefs.

(5) For any man always on the road, he wears the present invention not only just for personal hygiene and comfort purpose, but also for practical purpose when he does not carry enough clean briefs. In such case, he only has to replace the napkin of the present invention.

(6) To any man suffers hemorrhoids or particularly self-demanding personal hygiene, by having adhered the napkin inside his brief in a position relative to his anus will effectively help maintain a clean anus that may be left dirty after toilet or washing.

I claim:

1. A napkin for man's briefs napkin having its one end comprised of a tear-and-use adhesive side to be attached to a crotch inside the briefs and the other end a soft and absorbent napkin layer directly contacting the man's reproduction organ to absorb any residual urine after urination that may be left in external urethral orifice of the man's penis.

2. A napkin for man's briefs as claimed in claim 1, within, said napkin having its adhesive side attached inside the briefs at where contacts anus of the man.
